(19) United States
(12) Patent Application Publication
(10) Pub. No.: US 2014/0127287 A1
(43) Pub. Date: May 8, 2014

(54) LIPOSOME-ENCAPSULATED HYDROGELS FOR USE IN A DRUG DELIVERY SYSTEM

(71) Applicants: May Pang Xiong, Middleton, WI (US); Yan Wang, Madison, WI (US); Sheng Tu, Libertyville, IL (US)

(72) Inventors: May Pang Xiong, Middleton, WI (US); Yan Wang, Madison, WI (US); Sheng Tu, Libertyville, IL (US)

(73) Assignee: Wisconsin Alumni Research Foundation (WARF), Madison, WI (US)

(21) Appl. No.: 14/116,919

(22) PCT Filed: May 11, 2012
(86) PCT No.: PCT/US2012/037519

§ 371 Date: Jan. 13, 2014

Related U.S. Application Data
(60) Provisional application No. 61/484,811, filed on May 11, 2011.

(51) Int. Cl. A61K 9/127; A61K 31/395

(52) U.S. Cl. CPC A61K 9/127; A61K 31/395; A61K 9/1277
USPC 424/450; 514/183

(57) ABSTRACT

Drug delivery systems including a lipogel are disclosed. The lipogels allow for high drug loading and sustained release of drug molecules. Also disclosed are methods of making the drug delivery systems including lipogels.

### CROSS-REFERENCE TO RELATED APPLICATIONS

This application claims priority to International Application Number PCT/US2012/037519, filed on May 11, 2012, and U.S. Provisional Patent Application No. 61/484,811, filed on May 11, 2011, the disclosures of which are hereby expressly incorporated by reference in their entireties.

### STATEMENT REGARDING FEDERALLY SPONSORED RESEARCH OR DEVELOPMENT

This invention was made with government support under Grant Award No. CA136970 awarded by the National Institutes of Health. The government may have certain rights in the invention.

### BACKGROUND OF THE DISCLOSURE

The present disclosure relates generally to a novel drug delivery system including a liposome-encapsulated hydrogel and to methods for making the drug delivery system. More particularly, the drug delivery system includes an active agent encapsulated within a liposome-encapsulated hydrogel (i.e., lipogel), allowing for high drug loading and sustained release of the active agent.

Hydrogels are cross-linked networks of water soluble polymers that can absorb large amounts of water. Presence of water promotes biocompatibility and reduces cytotoxicity. The porous structure of hydrogels also allows for drug loading, making them advantageous for use as drug delivery systems.

Hydrogels that are pH sensitive tend to have a larger number of ionizable groups. The swelling ratio of these pH sensitive hydrogels depends on external pH. This on-off swelling state is due to electrostatic repulsion between polymer chains in the hydrogel network. For example, polyacrylic acid (“PAA”) has a large swelling ratio at higher pH due to the deprotonation of carboxylic groups and a small swelling ratio at lower pH due to protonation of carboxylic groups. PAA nanoparticles prepared in the past have been shown to increase in size from 70 nm to 120 nm when the pH was increased from 1 to 5. Bovine serum albumin (BSA) molecules were quantitatively loaded into PAA nanoparticles at pH<4.5 by taking advantage of electrostatic interactions between the anionic polymer and positive BSA (BSA is positively charged at pH<4.5).

The low tensile strength of hydrogels, however, limits their use as drug delivery systems due to the requirement for a long circulation time in the body. Previous studies have also shown that when hydrogels are used to load hydrophobic drugs, the leakage rate of drugs is generally very fast due to weak interactions between the hydrophilic polymer chains and hydrophobic drug molecules. For pH sensitive hydrogels, the pH change in the physiological environment also affects the loading and release of drugs from the gel. For example, loading of BSA molecules into PAA nanoparticles dropped to 20% when loaded at pH<7.4.

Furthermore, although surface modification of hydrogel nanoparticles can generally overcome the circulation disadvantage described above, it is difficult and time consuming to PEGylate or modify pre-made hydrogel nanoparticles. Modification of monomers for drug conjugation can also be complicated and can ultimately lead to limited drug loading.

Accordingly, there exists a need to develop alternative new methodologies that overcome the above-described disadvantages. Particularly, it would be advantageous for a drug delivery system that could allow for high loading and sustained release of drug molecules. Furthermore, it would be advantageous for the system to allow for easy surface modification such as PEGylation, addition of targeting ligands or antibody attachments.

### SUMMARY OF THE DISCLOSURE

It has been discovered that liposome-encapsulated hydrogels (also referred to herein as lipogel) may be useful as drug delivery systems for providing high drug loading and sustained release of drug molecules. Advantageously, the elastic-phospholipid bilayer shell of the system provides additional mechanical strength to the liposome-encapsulated hydrogel. Another advantage is that a low pH is maintained inside liposome-encapsulated hydrogels due to low proton exchange through the phospholipid bilayer that results in a small hydrogel swelling ratio. In turn, strong interactions may form between the hydrogel polymer and drug molecules to increase drug-loading. The lipid bilayer shell also allows for easy surface modifications such as PEGylation, addition of targeting ligands, or antibody attachments.

In one aspect, the present disclosure is directed to a drug delivery system comprising a lipogel and at least one active agent. In one particularly suitable embodiment, the present disclosure is directed to a drug delivery system comprising a lipogel and 17-(dimethylaminopropylamino)-17-demethoxygeldanamycin.

In another aspect, the present disclosure is directed to a method for preparing a drug delivery system. The method comprises: mixing a hydrogel precursor with a liposome to form a hydrogel precursor-liposome suspension; extruding the hydrogel precursor-liposome suspension; adding a free radical scavenger to the hydrogel precursor-liposome suspension; exposing the hydrogel precursor-liposome suspension to ultraviolet irradiation to polymerize the hydrogel precursor encapsulated by liposomes to form a lipogel; and incubating the lipogel with at least one active agent.

In yet another aspect, the present disclosure is directed to a method for preparing a drug delivery system. The method comprises: mixing a hydrogel precursor with a liposome to prepare a hydrogel precursor-liposome suspension; extruding the hydrogel precursor-liposome suspension; removing un-encapsulated hydrogel precursor from the hydrogel precursor-liposome suspension; treating the hydrogel precursor-liposome suspension to polymerize encapsulated hydrogel precursor to form a lipogel; and incubating the lipogel with at least one active agent.

### BRIEF DESCRIPTION OF THE DRAWINGS

The disclosure will be better understood, and features, aspects and advantages other than those set forth above will become apparent when consideration is given to the following detailed description thereof Such detailed description makes reference to the following drawing, wherein:

FIG. 1 is a graph depicting the in vitro release of 17-DMAPG from bulk gel, a liposome, and a liposome-encapsulated hydrogel as described in Example 2.

FIGS. 2A-2D are graphs depicting the size distribution of (A) liposomes (B) lipogels (C) liposomes mixed with Triton X-100 and (D) lipogels mixed with Triton X-100 measured using dynamic light scattering as discussed in Example 3.

FIG. 3 is a schematic illustrating the selective polymerization of hydrogel-forming materials inside liposomes as discussed in Example 3.

FIG. 4 depicts the polymerization reaction of acrylic acid and N,N′-methylenebis(acrylamide) initiated by UV irradiation that occurs within lipogels in the presence of ascorbic acid as discussed in Example 3.

FIGS. 5A-5C are graphs depicting the separation of nanogels from micelles by size exclusion chromatography as discussed in Example 3.

FIGS. 6A-6B are scanning electron micrographs of (A) lipogels (B) nanogels (with gold coating) and (C) liposomes as discussed in Example 3.

FIG. 7 is a graph depicting the loading of 17-DMAPG with lipogel under various pH as discussed in Example 4.

FIG. 8 is a graph depicting the loading of 17-DMAPG with lipogel under various temperatures as discussed in Example 4.

FIG. 9 is a graph depicting the loading of 17-DMAPG with lipogel under various incubation times as discussed in Example 4.

FIG. 10 is a graph depicting the loading of 17-DMAPG with lipogel under various drug:lipogel ratios (w/w) as discussed in Example 4.

FIG. 11 is a graph depicting the in vitro release of 17-DMAPG loaded in lipogel at pH 7.4 (Δ), liposome at pH 7.4 (⋄), bulk hydrogel at pH 7.4 (□), and bulk hydrogel at pH 3.0 (▪) as discussed in Example 5.

FIGS. 12A-12B are graphs depicting cytotoxicity of free 17-DMAPG and lipogel encapsulated 17-DMAPG on (A) PC-3 cells and (B) MDA-MB-231 cells as discussed in Example 6.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

In accordance with the present disclosure, compositions and methods have been discovered that are useful in a targeted drug delivery system and allow for high drug loading and sustained release of drug molecules.

### Liposome-Encapsulated Hydrogels

The present disclosure is directed to a liposome-encapsulated hydrogel (i.e., lipogels) for use in a drug delivery system. More particularly, the lipogel includes hydrogels encapsulated by a liposomal bilayer. Lipogels of the present disclosure combine hydrogel polymer biocompatibility with traditional liposome delivery properties to produce a new drug delivery system capable of high drug loading and sustained release. The liposomal bilayer of the lipogel not only provides mechanical and biological protection, but also helps maintain a low pH to allow for efficient loading of weakly basic active agents such as, for example, 17-dimethylaminopropylamino-17-demethoxy-geldanamycin (17-DMAPG). The strong charge-charge interaction between these polymers and active agents (e.g., drugs) also results in the slow active agent leakage observed in vitro, and thus allows for the preparation of sustained release of the active agent from the lipogel.

The liposomal bilayer may include any lipids or liposomes known in the art. The lipids make up one or more phospholipid bilayers that can act as a permeability barrier. Suitable lipids and liposomes may include, for example, dipalmitoylphosphatidylcholine, egg yolk L-α-phosphatidylcholine, 1,2-dimyristoyl-sn-glycero-3-phosphatidylcholine, 1,2-dipalmitoyl-sn-glycero-3-phosphatidylcholine, 1,2-dioleoyl-sn-glycero-3-phosphatidylcholine, 1,2-distearoyl-sn-glycero-3-phosphatidylcholine, 1,2-dilauroyl-sn-glycero-3-phosphatidylcholine, 1,2-dioleoyl-sn-glycero-3-phosphaethanolamine, 1-palmitoyl-2-oleoyl-sn-glycero-3-phosphaethanolaine, 1,2-dimyristoyl-sn-glycero-3-phosphaethanollamine, 1,2-dipalmitoyl-sn-glycero-3-phosphaethanolamine, 1,2-distearoyl-sn-glycerol-3-phosphaethanolamine, and combinations thereof. The phospholipid bilayer may be penetrable to an external stimulus such as UV, NIR/IR, temperature, and electrical stimuli.

Optionally, the lipids may be mixed with cholesterol to help stabilize the liposomal structure. Moreover, the cholesterol prolongs the circulation time of the liposomes within the human body. In general, a higher cholesterol level may increase the leakage rate of liposome-encapsulated contents. The cholesterol may be mixed with the lipids in a molar ratio of lipids to cholesterol of about 8:5, as this ratio has been found to be particularly suitable for liposome stability.

The lipogel additionally includes a hydrogel. The hydrogel includes water soluble hydrogel forming polymers and monomers for optimal encapsulation into the aqueous interior of the liposomes. The hydrogels vary and may be chosen based on desired properties for their compatibility with active agents. The hydrogels may further be modified with desired anionic, cationic, hydrophilic, or hydrophobic properties. Suitable hydrogels may be produced, for example, from pH sensitive hydrogel forming monomers. As used herein, “pH-sensitive” refers to monomers that can lose a proton to become negatively charged under basic pH conditions. Suitable pH sensitive hydrogel forming monomers may be, for example, anionic monomers such as acrylic acid, vinylsulfonic acid, 2-suloethylmethacrylate, 2-sulfoethyl acrylate, and combinations thereof, and cationic monomers such as 2-(dimethylamino)ethyl methacrylate, 2-(dimethylamino)ethyl acrylate, 2-(diethylamino)ethyl acrylate, and combinations thereof

Optionally, the hydrogels may include a cross-linker. Cross-linkers provide higher mechanical strength to the hydrogel. Cross-linking of the hydrogel polymers or monomers may also provide for a higher internal viscosity, slowing water exchange rate. Cross-linkers may be chosen such to provide polymer-polymer cross-linking or small molecule cross-linking. Small molecule cross-linkers are particularly preferred as more cross-linker may be included and the resulting hydrogel is more tightly packed, allowing for slower drug release. Suitable cross-linkers may include, for example, UV sensitive cross-linkers such as N,N′-methylenebis(acrylamide), poly(ethylene glycol)divinyl ether, and divinyl benzene. Other suitable UV sensitive cross-linkers include N,N′-(1,2-dihydroxyethylene)bisacrylamide, ethylene glycol diacrylate, di(ethylene glycol)diacrylate, tri(ethylene glycol)diacrylate, tetra(ethylene glycol)diacrylate, ethylene glycol dimethacrylate, di(ethylene glycol)dimethacrylate, tri(ethylene glycol)dimethacrylate, tetra(ethylene glycol)dimethacrylate, pentaerythritol triacrylate, and combinations thereof. Temperature sensitive cross-linkers such as diisoyanates (e.g., macrodiisoyanates (MDICs)) are also suitable for use in the hydrogels of the lipogels of the present disclosure.

Typically, the hydrogels include one or more cross-linkers in an amount of from about 1% to about 15% by weight hydrogel. It should be recognized, however, that the greater amounts of cross-linkers used with the hydrogels, the higher the viscosity and lower the elasticity of the hydrogel. Accordingly, the amount of cross-linker may be adjusted depending on the desired resulting hydrogel viscosity and/or elasticity.

Optionally, the hydrogel may further include water soluble and/or partially water soluble initiators. Suitable initiators include, for example, photoinitiators and thermo-sensitive initiators including, for example, 2,2-diethoxyacetophenone, 2,2-dimethoxy-2-phenylacetophenone, di-t(tertiary)-butylperoxide, azobisisobutyronitrile, 1-[4-(2-hydroxyethoxy)phenyl]-2-hydroxy-2-methyl-1-propan-1-one, 2-hydroxy-2-methylpropiophenone, and 2-hydroxy-4′-(2-hydroxyethoxy)-2-methylpropiophenone, and redox initiators including, for example, ammonium persulfate, potassium persulfate, 1,1′-azobis(cyclohexanecarbonitrile), 2,2′-azobis 2-methyl-N-(2-hydroxyethyl)propionamide, 2,2′-azobis(2-amidinopropane)dihydrochloride, 4,4′-azobis(4-cyanovaleric acid), and combinations thereof

The initiators may be used to control the time of cross-linking of the hydrogel polymers and monomers. This may allow for longer term storage of the lipogels and their contents so that degradation/deformation of the lipogel is minimized

Typically, initiators may be included in the hydrogel in an amount of from about 0.5 mol % to about 10 mol %, including from about 0.75 mol % to about 5 mol %, and including about 1 mol %.

The lipogels may vary in size depending on the active agent to be encapsulated within the hydrogel of the lipogel and the end use of the lipogel. Suitable lipogel diameter may be from about 100 nm to about 400 nm. Particularly suitable lipogel diameter may be from about 100 to about 200 nm. The desired lipogel diameter may be obtained using membranes (e.g., polycarbonate membranes) having a pore size corresponding to the desired lipogel diameter. Thus, for example, if a lipogel having a diameter of about 120 nm is desired, a membrane having a pore size of about 120 nm may be used.

In another aspect, lipogels of the present disclosure may be surface modified. Suitable surface modifications may be, for example, PEGylation, target ligand attachment, and antibody attachment. Modification of the lipogels may, for example, prolong circulation time of the lipogel within the body. Further, modification such as PEGylation may help evade attack of the lipogel by the reticular-endothelial system (RES) in the human body.

The amount of targeting ligands for surface modification in the lipogels depends on the type and affinity levels of the desired target receptors. In one suitable embodiment, the lipogels include about 20 mol % targeting ligand levels.

### Drug Delivery Systems

As noted above, the present disclosure is directed to a drug delivery system including the above-described lipogel. More particularly, the drug delivery system includes a lipogel and at least one active agent.

Suitable materials for use in producing the lipogels may be any of the lipids/liposomes, hydrogels, cross-linkers, initiators, and any other optional components described herein.

The drug delivery system also includes at least one active agent. Suitable active agents may be, for example, a drug, a nutraceutical, and/or a pharmaceutical agent. The active agent may vary depending on the polymers and/or monomers for use in the hydrogel of the lipogel. For example, weakly basic active agents are suitable for use in the lipogels formed from anionic hydrogel-forming monomers. As used herein, “weakly basic” refers to agents including molecules with functional groups having a pKa of about 7 to about 10. Examples of weakly basic molecules include amines, which can protonate at low pH levels, and thus, can carry a positive charge. Alternatively, the hydrogel polymers and monomers can be tailored for use with weakly acidic molecules. As used herein, “weakly acidic” refers to agents including molecules with functional groups having a pKa of from about 2 to about 5.

Suitable active agents may include, for example, anionic molecules such as valproic acid (pKa 4.8), aspirin (pKa 3.5), and penicillin (pKa 2.7), cationic molecules such as doxorubicin (pKa 8.3), 17-(dimethylaminopropylamino)-17-demethoxygeldanamycin (17-DMAPG) (pKa 7.6), and opioids (e.g., hydromorphone (pKa 8.1)), and amphiphilic molecules such as cisplatin (pKa 6.6), vincristine (pKa 5.0 and 7.4), mitoxantrone (pKa 6.0 and 8.1), ciproflaxin (pKa 6.0 and 9.0), vinorelbine, (pKa 5.4 and 7.4), and topotecan (pKa 10.5, pKa 7.0, and pKa 0.6).

Additional active agents may include proteins such as bovine serum albumin, insulin, p53 tumor suppressor protein, and growth factors. Still other suitable active agents include genes such as pDNA, dsRNA, and siRNA.

Active agent loading in the drug delivery system may be greater than 60%. More suitably, active agent loading may be greater than 70%. Even more suitably, active agent loading may be greater than 80%. Even more suitably, active agent loading may be greater than 90%.

Another aspect of the drug delivery system is sustained release of the active agent. For example, the release of the active agent from the lipogels may be about 40% over a range of about 24-hours to one week period as compared to conventional hydrogels and liposomes, which typically release 90% of the active agent within 5 hours.

The drug delivery systems release the active agents using one of three mechanisms: diffusion-controlled release, swelling-controlled release, and chemically-controlled release. Diffusion-controlled release is the most dominant mechanism and depends on interactions between the hydrogel and the active agent as well as the porous structure of the lipogel. More particularly, the interactions between the ionic polymer matrix and the active agent molecules and the high viscosity of the cross-linked hydrogel matrix within the lipogel help to lower the diffusion rates through the lipogel. Accordingly, the diffusion rate can be modified and controlled by modifying the active agent molecule-polymer interactions and/or the hydrogel internal viscosity.

Swelling-controlled release occurs when the drug diffusion rate is faster than the hydrogel swelling rate. For example, nano-sized lipogels such as polyacrylic acid (PAA) nanoparticles have very high swelling rates. Chemically-controlled release relies on chemical or pH changes in the environment to initiate the release of the active agent from the lipogel.

### Methods for Preparing Drug Delivery Systems Including Lipogels

Another aspect of the present disclosure is directed to a method of preparing the drug delivery systems including the lipogels. In one aspect, the method includes mixing a hydrogel precursor with a liposome to prepare a hydrogel precursor-liposome suspension; extruding the hydrogel precursor-liposome suspension; removing un-encapsulated hydrogel precursor from the suspension; treating the suspension to polymerize encapsulated hydrogel precursor to form a lipogel; and, once the lipogel is formed, incubating the lipogel with at least one active agent.

Initially, in some embodiments, a hydrogel precursor may be prepared using an aqueous solution including the water soluble hydrogel-forming polymers and/or monomers described above. In some suitable embodiments, the solution includes hydrogel-forming monomers, a cross-linker and an initiator as described above. For example, in one particular embodiment, the solution includes approximately 100 mg/ml hydrogel-forming monomer, 10 mg/ml cross-linker, and 1 mg/ml of initiator.

The hydrogel precursor is mixed with a liposome as described above to form a suspension of the hydrogel precursor and the liposome (i.e., hydrogel precursor-liposome suspension). In one suitable embodiment, the liposome is in the form of a liposome film as the film allows for improved hydration when forming the lipogels. To form the liposome film, lipids are first dissolved in chloroform and then an organic solvent is used for rotary evaporation, leaving a thin, porous film. The lipid film is then hydrated with a solution such as, for example, hydrochloric acid. The solution may further include the hydrogel precursor.

In one embodiment, the lipid or liposome is mixed with cholesterol as described above prior to being mixed with the hydrogel precursor.

The suspension is then extruded using a suitable extruder as known in the art. In one embodiment, the extruder is a mini syringe extruder, such as commercially available from Avanti Polar Lipid (Alabaster, Ala.). Extrusion of liposomes encapsulating the hydrogel precursor (hydrogel monomer and/or polymer, cross-linker and initiator) from the suspension through a membrane having a particular pore size allows for the preparation of liposomes having a desired hydrodynamic diameter. Suitable membranes may be, for example, polycarbonate membranes having a pore size of from about 100 nm to about 400 nm. Suitable particle sizes may be from about 80 nm to about 120 nm, including about 100 nm.

Extrusion may be conducted at a temperature of approximately 30° C. to 70° C., including from about 40° C. to about 60° C., and including about 55° C.

The suspension contains hydrogel precursor that is encapsulated by liposomes and un-encapsulated hydrogel precursor surrounding the liposome encapsulate. The suspension is then subjected to further processing to remove un-encapsulated hydrogel precursors from the suspension. Suitable methods may be, for example, processing the extruded suspension including the liposome suspected of containing un-encapsulated hydrogel by column chromatography. For example, in one embodiment, the extruded suspension is subjected to a Sephadex G 50 size exclusive column with 10 mM HCl solution as the elution buffer. Another suitable isolation method includes ultracentrifugation, which causes the liposomes encapsulating hydrogel precursor to settle as pellets, thereby leaving un-encapsulated hydrogel precursor in the supernatant.

Further, the method includes polymerization of the suspension of encapsulated hydrogel precursor (including hydrogel-forming monomers), which may be initiated using any treatment known in the art for polymerizing monomers. In one embodiment, the suspension is treated with an ultraviolet (UV) irradiation treatment to cause polymerization of the monomers. For example, the suspension including the encapsulated hydrogel monomers is treated with ultraviolet light having a wavelength of approximately 365 nm for a period of from about 1 minute to about 30 minutes, including from about 2 minutes to about 15 minutes, and including from about 3 minutes to about 10 minutes. In one particularly suitable embodiment, the aqueous solution is treated with UV light at a wavelength of 365 nm for a period of 3 minutes using a 160 W Blak-Ray high intensity UV lamp.

In another aspect, the present disclosure is directed to a method of preparing the drug delivery systems including the lipogel, wherein any un-encapsulated hydrogel precursor is not removed from the suspension prior to hydrogel polymerization. The method of this aspect includes forming a hydrogel precursor-liposome suspension of a hydrogel precursor with a liposome; extruding the hydrogel precursor-liposome suspension; adding a free radical scavenger to the suspension; treating the hydrogel precursor-liposome suspension to polymerize the hydrogel precursor encapsulated by the liposome to form a lipogel; and, once the lipogel is formed, incubating the lipogel with at least one active agent.

The hydrogel precursor is mixed with a liposome to form a hydrogel precursor-liposome suspension and extruded as described above. As previously noted, the suspension contains hydrogel precursor that is encapsulated by liposomes and un-encapsulated hydrogel precursor surrounding the liposome encapsulate. The method further includes adding a free radical scavenger to the suspension. Suitable free radical scavengers may be, for example, ascorbic acid, uric acid, glutathione, melatonin, vitamin E, and combinations thereof. The suspension containing the free radical scavenger is then treated to polymerize the hydrogel precursor encapsulated by the liposome to form a lipogel. Suitable treatments to polymerize the hydrogel precursor encapsulated by the liposome to form a lipogel may be, for example, ultraviolet irradiation and heat.

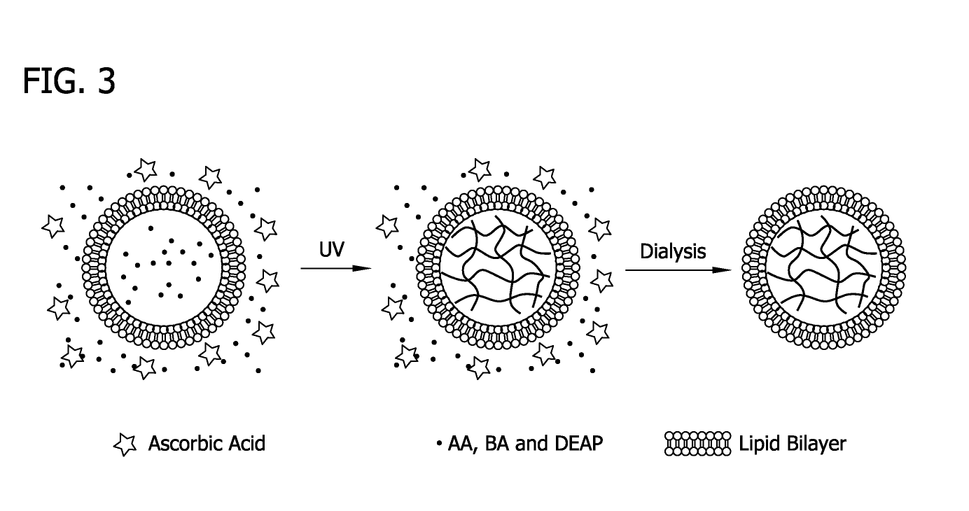

Without being bound by theory, the presence of excess free radical scavenger in the suspension may impede the polymerization of un-encapsulated hydrogel precursors in the aqueous solution exterior to the liposomes. In the interior liposomal compartment that lacks free radical scavenger, however, polymerization can occur to result in a cross-linked hydrogel matrix with a boundary defined by the surrounding liposome (see, FIG. 3).

Once the lipogel is formed, the method further includes incubating the lipogel with at least one active agent. Incubating the lipogel with a solution containing an active agent allows for the active agent to be incorporated into the hydrogel portion of the lipogel. In one embodiment, the lipogel is incubated with the active agent in an incubator at a temperature from about 37° C. to about 65° C. for about 24 hours. Particularly suitable incubation temperatures may be from about 45° C. to about 55° C. In another embodiment, the lipogel is incubated with the active agent wherein the solution containing the active agent has a pH from about 4 to about 7.4. Suitable incubation times may be from about 5 minutes to about 24 hours. Particularly suitable incubation times may be from about 5 minutes to about 60 minutes. Suitable active agent to lipogel ratio (w/w) for loading may be from about 0.01 to about 0.1.

The disclosure will be more fully understood upon consideration of the following non-limiting Examples.

### EXAMPLES

### Example 1

In this Example, lipogels of the present disclosure were prepared.

Specifically, bulk hydrogel precursors were prepared by ultraviolet-initiated polymerization of aqueous solutions of acrylic acid (AA; Sigma-Aldrich), N,N′-methylenebis(acrylamide) (BA; Sigma-Aldrich) as a cross-linker, and 1% w/v 2,2-diethoxyacetophenone (DEAP; Sigma-Aldrich) as an initiator. The sample was treated with a 100 W Blak-Ray high intensity UV lamp (Mineralogical Research Co, CA) for 90 seconds. Large unilamellar liposomes were prepared with dipalmitoylphosphatidylcholine (DPPC) and cholesterol (Chol) in a 3:1 molar ratio using direct hydration and extrusion. Particularly, a liposome film was hydrated with 1 mL 10 mM HCl solution containing the hydrogel precursors. The resulting hydrogel precursor-liposome suspension was extruded through a micro-extruder (Avanti Polar Lipid) equipped with a 100 nm pore size polycarbonate filter.

The extruded suspension includes the liposomes having a unimodal size distribution of 100 nm to 120 nm, as observed with dynamic light scattering (DLS). Extruded liposomes were passed through a Sephadex G 50 column to remove un-encapsulated precursors that could polymerize outside liposomes in solution. Liposomes loaded with internal hydrogel precursors were then treated with UV radiation for 3 minutes to initiate polymerization, yielding the lipogels.

### Example 2

In this Example, drug loading and release from lipogels was compared to drug loading and release from liposomes and un-encapsulated hydrogels.

Lipogel and liposome preparations prepared according to Example 1 were incubated in 1 mL 17-DMPAG drug solution (pH 6.5 HEPES buffer). After 6 hours incubation at room temperature, samples were passed through a Sephadex G50 column to remove unincorporated free drugs. The drug-loaded lipogels and drug-loaded liposomes were dissolved in 2-Propanol before HPLC quantitation. In vitro drug release studies were conducted by dialyzing drug-loaded lipogels or drug-loaded liposomes against 400 mL pH 6.5 HEPES buffer.

Bulk hydrogel (un-encapsulated hydrogel) prepared according to Example 1 was incubated in 1 mL 17-DMPAG drug solution (pH 6.5 HEPES buffer). After 6 hours, samples were centrifuged to remove unloaded free drugs. The drug concentration in the supernatant was analyzed to determine drug loading percentage. The drug loaded-hydrogel was then put into a 400 mL pH 6.5 HEPES buffer solution for in vitro release studies.

Bulk Hydrogel Loading and Release

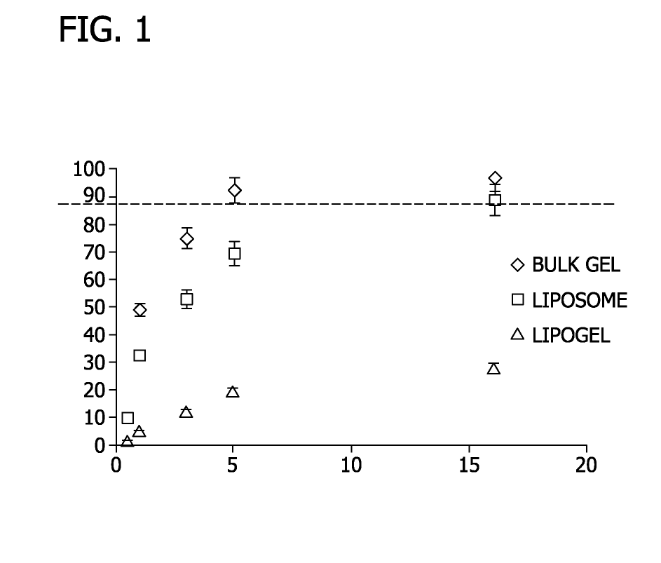

17-DMPAG was efficiently loaded into bulk hydrogel (over 60% after 6 hours room temperature incubation). This is because the original pH (pH 2) of the bulk gel immediately increased to 6.5 when the hydrogel was incubated in drug solution (pH 6.5 HEPES buffer). Thus, carboxylic acid side chains of PAA gel became deprotonated and negatively charged at pH 6.5. Similarly, 17-DMPAG molecules are weakly basic (pKa around 9) and therefore approximately 99% become protonated and positively charged at pH 6.5. This ionic interaction between carboxylates and cationic drugs is the driving force for high drug loading into hydrogels. Although drug loading was considerably high in the bulk gel, the higher pH also increased its swelling ratio. This caused higher molecular mobility of drug molecules within the porous gel matrix and resulted in rapid drug leakage (FIG. 1).

Liposome Drug Loading and Release

Liposomes with low internal pH have been widely used in the past to achieve efficient loading of weakly basic molecules. This method takes advantage of the low lipid bilayer permeability of ions. Neutral base molecules have higher lipid bilayer permeability than charged forms, and therefore, can easily diffuse into the liposome. Once neutral drug molecules diffuse into a liposome, they pick up protons due to the lower internal pH. The positively charged drug ion remains trapped in the liposome and this gradient drives more neutral drugs into the liposome. This method of drug loading is not efficient when very hydrophobic drugs are used due to their high permeability through the lipid bilayer. 17-DMPAG is a weakly basic molecule with high hydrophobicity (logP 4.6). Thus, the loading efficiency of 17-DMAPG into extruded DPPC/Chol liposomes with low pH was relatively low (−15%) and exhibited fast leakage (FIG. 1).

Lipogel Drug Loading and Release

At low internal pH of the liposome, carboxylic acid side chains of PAA become protonated. Neutral 17-DMPAG drug molecules diffused through the lipid bilayer and into the gel. These neutral drug molecules then picked up a proton from carboxylic acid side chains and formed stable ion-pairs. This drives more neutral drug molecules into the lipogel. Since the lipid bilayer blocks proton and water exchange with the external medium, it is able to maintain its low internal pH to keep the gel more viscous and in a more de-swollen state. As a result, quantitative drug loading (100%) into lipogels was obtained and in vitro release studies demonstrated very slow release of drugs over 24 hrs (FIG. 1).

### Example 3

In this Example, lipogels of the present disclosure were prepared.

Sodium ascorbate, acrylic acid (AA), N-isopropyl acrylamide (NIPAm), N,N′-methylenebis(acrylamide) (BA), 2,2-diethoxyacetophenone (DEAP), 3-(dimethylamino)-1-propylamine and Triton X-100 were purchased from Sigma-Aldrich. Geldanamycin was obtained from LC Solutions. Thin-layer chromatography was performed using DC-Alufolien Kieselgel 60 F254 plates (EMD Chemicals, Darmstadt, Germany). Visualization was achieved by UV light (254 nm) and with ceric molybdate stain activated by heat. For flash chromatography, silica gel 40-63 μm from EMD Chemicals (Darmstadt, Germany) was used. 1H NMR spectra were acquired on Varian Inova 500 spectrometer. Chemical shifts are reported in parts per million (ppm) relative to tetramethylsilane, and spin multiplicities are given as s (singlet), d (doublet), t (triplet), q (quartet), m (multiplet), or br s (broad singlet). High-resolution mass spectra were obtained on an IonSpec 7 Tesla HiResMALDI FT-Mass Spectrometer at the Analytical Instrumentation Center of University of Wisconsin School of Pharmacy. 1,2-Dipalmitoyl-sn-glycero-3-phosphocholine (DPPC) and cholesterol were purchased from Avanti Polar Lipids. Dulbecco's Modified Eagle Medium (DMEM), fetal bovine serum (FBA) and 1% penicillin/streptomycin were purchased from Cellgro.

To prepare lipogels, 80 μmol DPPC and 50 μmol cholesterol were dissolved in chloroform. The solvent was evaporated on a rotary evaporator under vacuum. The dried lipid film on the flask was rehydrated with 1 ml of 10 mM HCl containing the hydrogel precursors (100 μl AA, 10 mg BA and 1 μl DEAP) in a 55° C. water bath, with vigorous shaking for 30 min. The resulting multilamellar vesicles were extruded 11 times through a 100 nm polycarbonate filter via a mini-extruder (Avanti Polar Lipids), generating large unilamellar vesicles (LUV).

Sodium ascorbate was added to the solution at a molar ratio of 200:1 relative to DEAP. After 5 min of UV irradiation (Black Ray UV lamp, 365 nm, 100 Watt), polymerization of monomers in liposomes was completed and lipogels of ca. 100-120 nm diameter were obtained. Polymerization outside liposomes was impeded using ascorbic acid as a scavenger of free radicals. The lipogel suspension was then dialyzed against deionized water to remove un-encapsulated hydrogel precursors and ascorbic acid molecules. Lipogels were concentrated via ultracentrifugation and resuspended in desired buffer.

To separate the lipid bilayer from the bare hydrogel nanoparticles, Triton X-100 was mixed with lipogels at a 50:1 surfactant/lipid molar ratio. The solution was then heated to 90° C. until it became cloudy (as an indication that the solution reached the cloud point of the Triton X-100) and allowed to cool to room temperature. DPPC and cholesterol formed micelles with Triton X-100, leading to removal of the bilayer from the hydrogel core. This mixture was then eluted through a PD-10 column (used as a size exclusion chromatography column) to separate micelles from the resulting hydrogel nanoparticles. Fractions were collected and analyzed by dynamic light scattering.

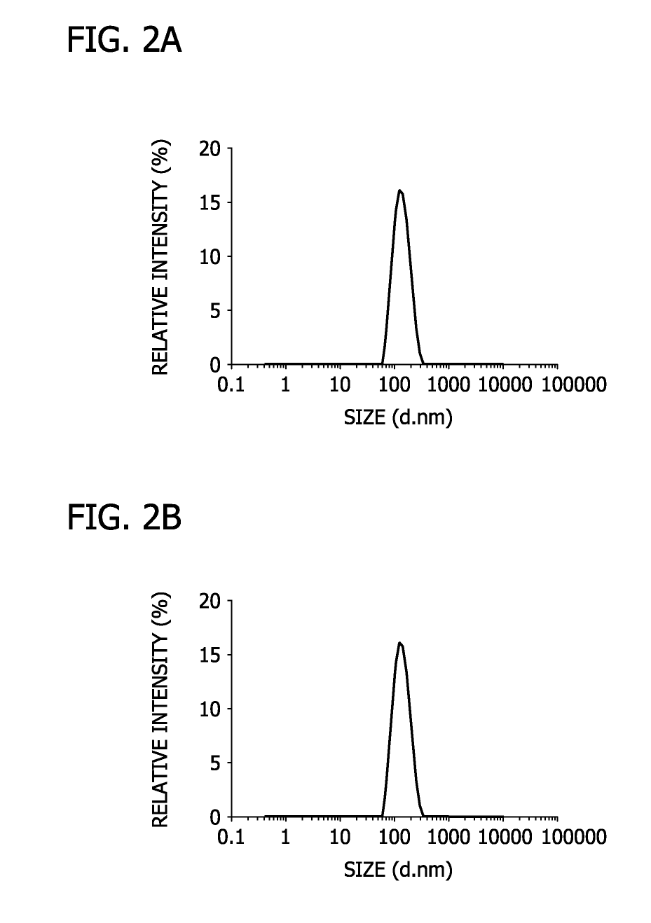

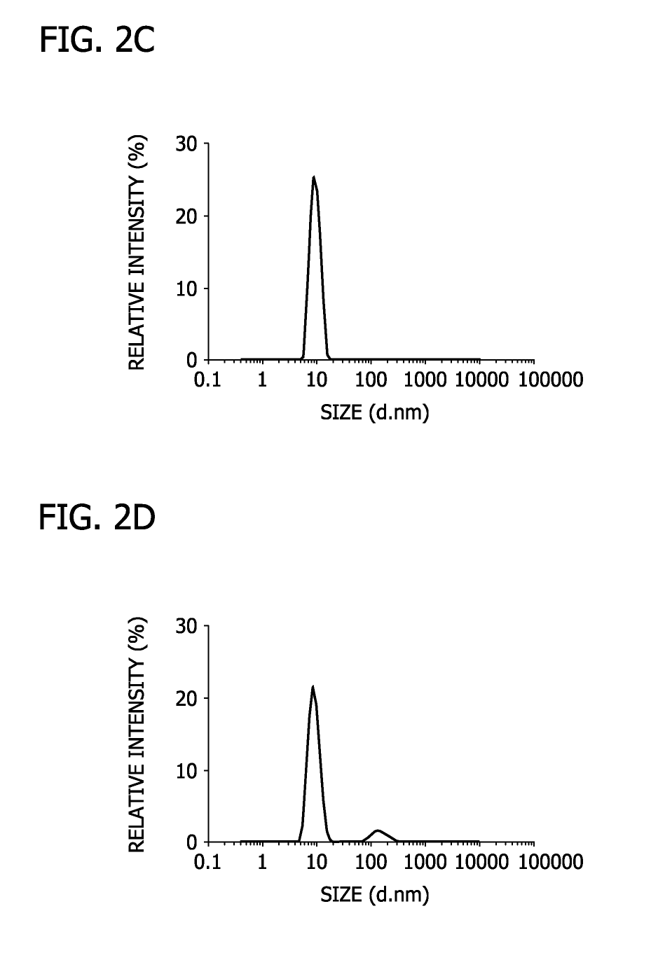

Liposomes encapsulating AA (as monomers), BA (as cross-linker) and DEAP (as photo-initiator) were prepared using the standard thin-film hydration method followed by repeated extrusion through polycarbonate membranes with pore sizes of 100 nm. As shown in FIG. 2A, the hydrodynamic diameter of liposomes was ˜120 nm with low polydispersity. As precursor of lipogels, the liposomes defined the boundary of lipogels, which had similar size distribution with liposomes (FIG. 2B). The control of the lipogel size by extrusion allowed us to prepare lipogels with different sizes, e.g. ˜200 nm and ˜400 nm by using polycarbonate membranes with different pore size. Here we chose lipogels with diameter ca. 120 nm as a model system.

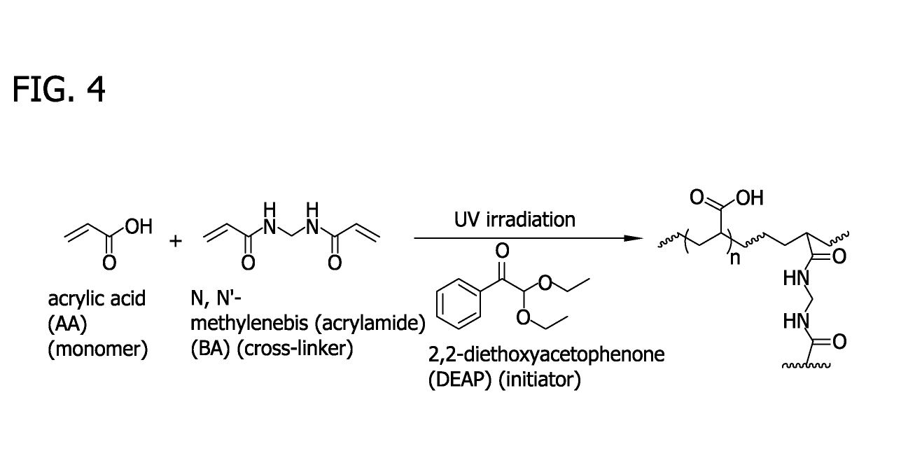

Following the preparation of liposomes, lipogels were formed by selective cross-linking of hydrogel-forming materials inside liposomes. As is shown in FIG. 3, un-encapsulated AA/BA/DEAP were not separate from liposomes after extrusion, but rather ascorbic acid was added into the mixture. As a free radical scavenger, polymerization was greatly impeded in the exterior aqueous solution of liposomes in the presence of large quantity of excess ascorbic acid. However, in the liposomal interior compartment, there was no ascorbic acid. Thus, the UV-initiated polymerization resulted in cross-linked PAA hydrogel matrix with a defined boundary. The polymerization chemistry is depicted in FIG. 4. Un-encapsulated components were removed by dialysis against deionized water. Similar size distribution of lipogels and liposomes indicates that gelation inside liposomes did not change the hydrodynamic diameter (FIGS. 2A and 2B).

To confirm the gelation inside liposomes, the lipid bilayer on lipogels was removed with Triton X-100 and Dynamic Light Scattering (DLS) was used to monitor the bare nanogels inside liposomes and the size distribution of nanoparticles. The cumulant diameters of samples were calculated by the Stokes-Einstein equation and sizes are reported as Z-average diameters. For ζ-potential measurements, samples (800 μL) in 10 mM Tris-HCl buffer (pH 7.0) were added to a capillary cell and the electrophoretic mobility was measured. ζ-potential was calculated by the Henry equation. Both DLS and ζ-potential measurements were taken with the Zetasizer Nano ZS (Malvern Instruments).

In the presence of excess micelle-forming Triton X-100, DPPC and cholesterol is preferentially incorporated into the micelles while bare nanogels were exposed to external aqueous solution, leaving a ˜200 nm peak on DLS graph (FIG. 2D). The slight increase of hydrodynamic diameter from lipogels (ca. 120 nm) to bare nanogels (ca. 200 nm) was probably due to the swelling of hydrogel matrix. Once the lipid bilayer has been removed, the PAA hydrogel matrix was exposed to a higher pH and de-protonation on carboxylic acid groups resulted in higher expulsion force among polymer chains. As a comparison, liposomes encapsulating AA/BA/DEAP, but not polymerized by UV irradiation were also mixed with Triton X-100. Upon addition of Triton X-100, the ˜120 nm peak of liposomes completely disappeared with the emergence of ˜10 nm micelle peak (FIG. 2C).

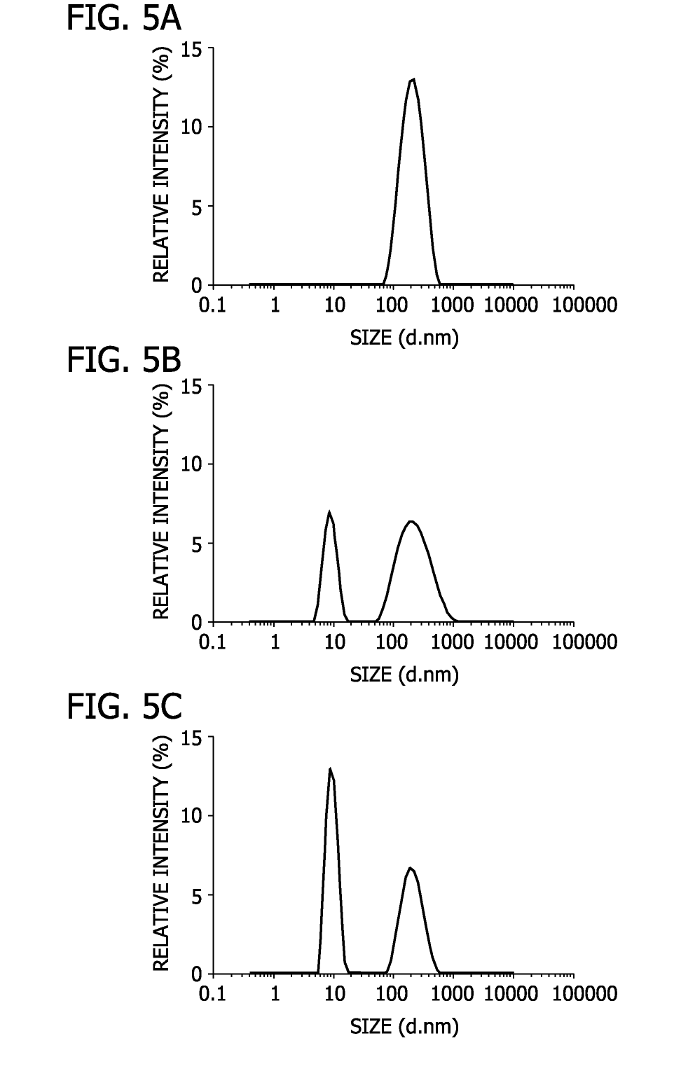

To obtain pure hydrogel nanoparticles for further characterization, some of the 200 nm nanogels were partially separated from 10 nm micelles by size exclusion chromatography (SEC). The first collected fraction contained pure nanogels (FIG. 5A), while subsequent fractions had increasingly more 10 nm micelles (FIGS. 5B and 5C). The bare nanogels as well as liposomes and lipogels were then dried on silicon substrates and characterized by scanning electron microscopy (SEM). For scanning electron microscopy (SEM), nanoparticle samples were dispersed in deionized water and dropcasted onto silicone (Si) substrates and briefly air dried. The substrates were then mounted onto metallic pucks using double-sided carbon tape and imaged with a LEO Supra 55 VP field emission SEM. In the case of bare hydrogel nanoparticles, the sample was sprayed with a layer of ˜20 nm gold before observation with SEM.

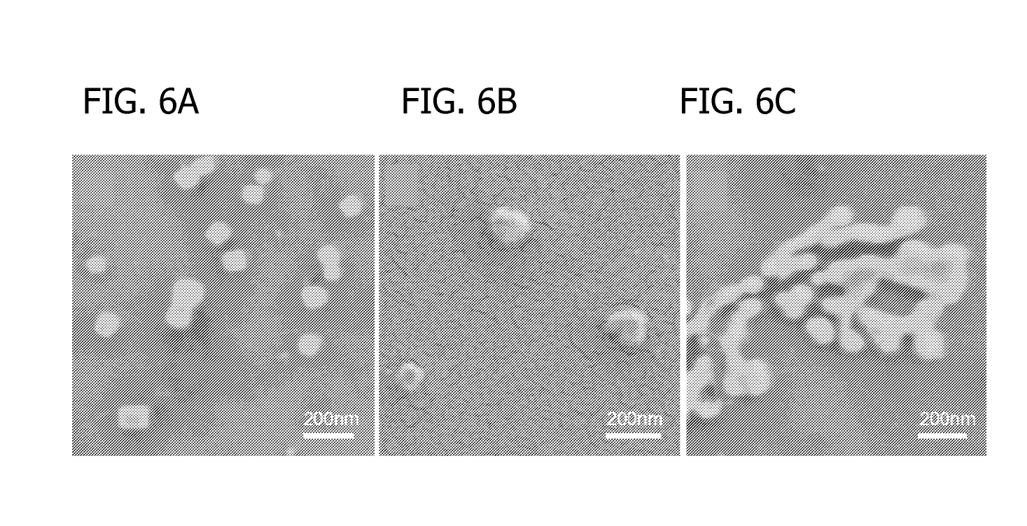

Interestingly, both lipogels (FIG. 6A) and bare nanogels (FIG. 6B) were spherical and toroidal shaped as expected, while liposomes appeared like merged clusters (FIG. 6C). Liposomes prepared by repeated extrusion were usually spherical shaped in aqueous solution, but their structure may collapse and merge upon drying out in the vacuum chamber of SEM due to lack of structural support from inside. On the contrary, lipogels had the structural support from interior polymer matrix, so they did not collapse upon drying out. This result indicated the enhanced physical strength of lipogels compared with conventional liposomes. As to bare nanogels, their mass density was much lower than lipogels with the removal of lipid bilayer, so the sample was coated with ˜20 nm gold in order to clearly observe them under SEM otherwise they appeared too pale. The zeta potential of these nanoparticles was also measured. As expected, empty liposomes had the most neutral charge at 0.19 mV, lipogels were characterized with a −39 mV charge and bare hydrogel nanoparticles had a −58 mV surface charge.

### Example 4

In this Example, lipogels of the present disclosure were loaded with 17-DMAPG.

17-DMAPG was obtained by one-step reaction from geldanamycin with as high as 95% yield. Lipogels prepared as described in Example 3 were incubated with 17-DMAPG in pH 7.4 PBS buffer and incubated in a 55° C. water bath for 30 min. After cooling to room temperature, un-encapsulated drug was then removed by eluting the suspension through a PD-10 column. To determine the concentration of encapsulated 17-DMAPG, isopropyl alcohol (IPA) was used to rupture the lipid bilayer and to release 17-DMAPG from the hydrogel core. The released 17-DMAPG in the IPA solution was quantified by reverse phase HPLC. For in vitro release studies, 3 mL of the drug loaded lipogel suspension was injected into a Slide-A-Lyzer dialysis cassette (10,000 MWCO, Thermo Scientific) and dialyzed against 1 L pH 7.4 PBS buffer. Then, at indicated time points 50 μL aliquots were withdrawn from the cassettes for HPLC analysis.

17-DMAPG was first loaded into bulk PAA hydrogels to verify that the cationic drug binds to anionic PAA polymer matrix. Bulk hydrogels were prepared by UV initiated polymerization of 10% AA and 1% BA aqueous solution containing 0.1% DEAP as photo initiator. When 1 ml bulk hydrogel was immersed in 1 ml of 2 mg/ml 17-DMAPG solution (used deionized water as solvent) and incubated at room temperature, the concentration of 17-DMAPG in the outer solution kept decreasing as determined by HPLC. There was no further decrease after 10 hours, at which time there was 11% 17-DMAPG remaining in the outer solution, meaning that 89% of 17-DMAPG was loaded into the bulk hydrogel. This was in contrast to PNIPAm hydrogel prepared from 10% NIPAm and 1% BA, which only loaded 60% of 17-DMAPG under the same conditions. Since both PNIPAm and PAA hydrogels were prepared with the same cross-linking density, the higher loading efficiency of 17-DMAPG into PAA hydrogel was attributed to the ionic interaction between cationic drug and carboxylic acids grafting on the polymer matrix.

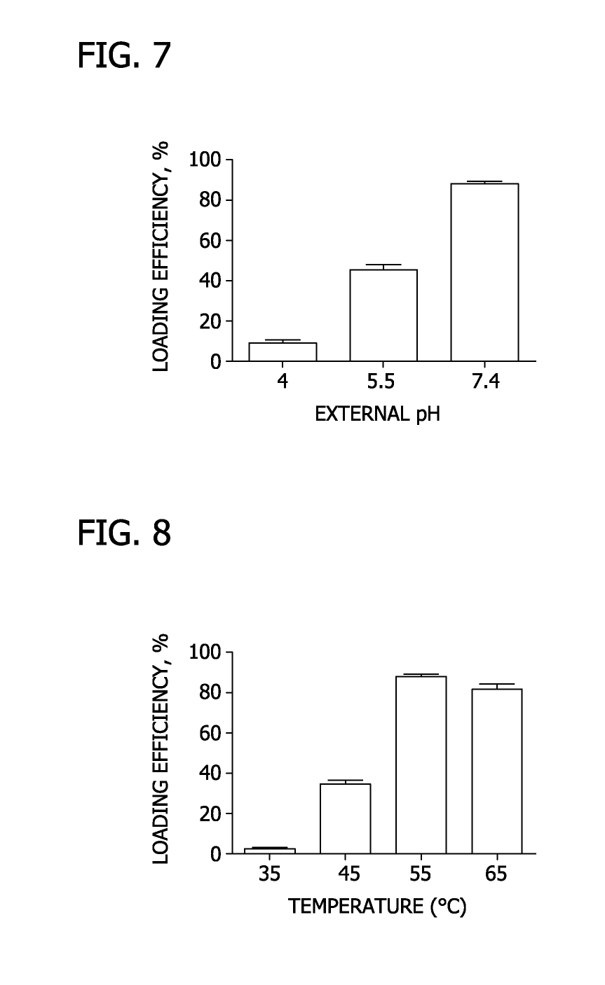

Similar to the remote loading of liposomes which commonly utilizes a pH gradient, an external pH of 7.4 was found to effectively load 17-DMAPG into lipogels. Assuming that polymerization and gelation did not change the pH of 10% acrylic acid solution, the pH inside lipogel cores would be around 1.0. As shown in FIG.7, when the external pH of incubation solution increased from 4.0 to 7.4, drug loading increased from 9% to 88%, indicating that pH gradient is favorable for lipogel drug loading.

Incubation temperature also had an impact on the loading efficiency. Lipid bilayers had a transition temperature at which they underwent transition from solid “gel” phase to liquid crystalline as temperature increased, whereas in the latter phase, lipids were able to freely diffuse within the two-dimensional plane of the membrane, and the permeability for many drug molecules were much higher. Although DPPC had a transition temperature of 41° C., the incorporation of about 38 mol % cholesterol into lipid bilayer complicated the situation. As shown in FIG.8, loading efficiency was nearly zero at 35° C., and continuously increased from 45° C. to 55° C., so it is likely that the phase transition temperature of the system was reached around 45-55° C. Slight decreased loading efficiency at 65° C. was possibly due to the heat instability of 17-DMAPG, where another peak was observed by HPLC analysis, indicating a fair amount of transformation of 17-DMAPG at extended incubation under high temperature.

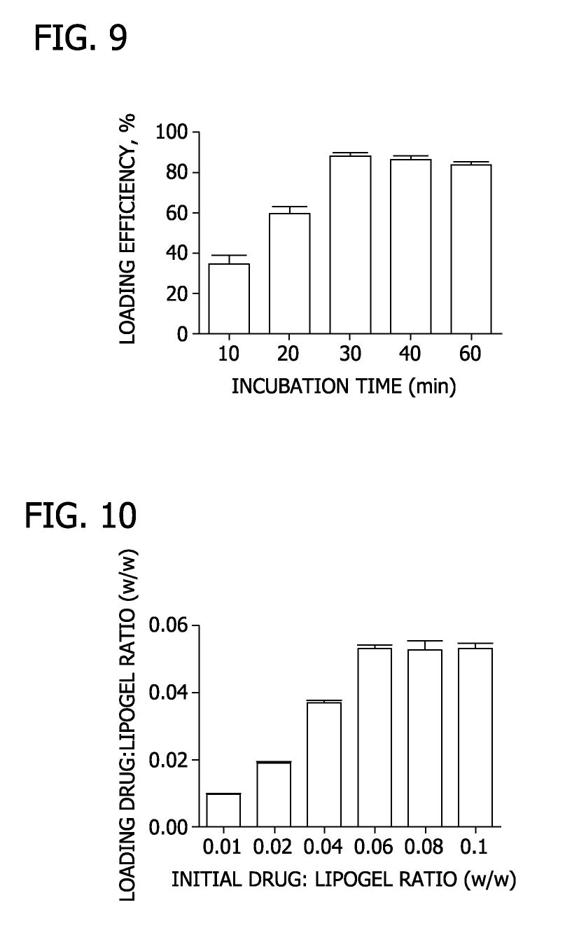

Loading efficiency was also monitored with incubation time length (FIG. 9). A maximum loading capacity was reached at 30 min incubation under 55° C., while a slight decrease on loading efficiency, probably due to drug decomposition, occurred with extended incubation time.

Drug loading capacity was found to be about 0.053 mg 17-DMAPG per 1 mg lipogel (FIG. 10). As the drug:lipogel ratio (w/w) was varied from 0.01 to 0.1 with fixed concentration of lipogel, the concentration of loaded drug reached a plateau above 0.06. A drug loading percentage of 88% or higher was obtained at a drug:lipogel ratio below 0.06.

To demonstrate the advantage of lipogel systems of the present disclosure over plain liposomes, liposomes encapsulating 10 mM HCl that had an acidic inner aqueous compartment were prepared. The liposomes were incubated with 17-DMAPG under the same conditions described above (pH 7.4, 55° C. and 30 min) Using the same amount of drug and lipid where the drug:lipogel ratio (w/w) was 0.06, the plain liposomes loaded only about 10% of 17-DMAPG, which was in sharp contrast to the 88% loading with lipogels of the present disclosure.

### Example 5

In this Example, drug release from lipogels of the present disclosure was determined

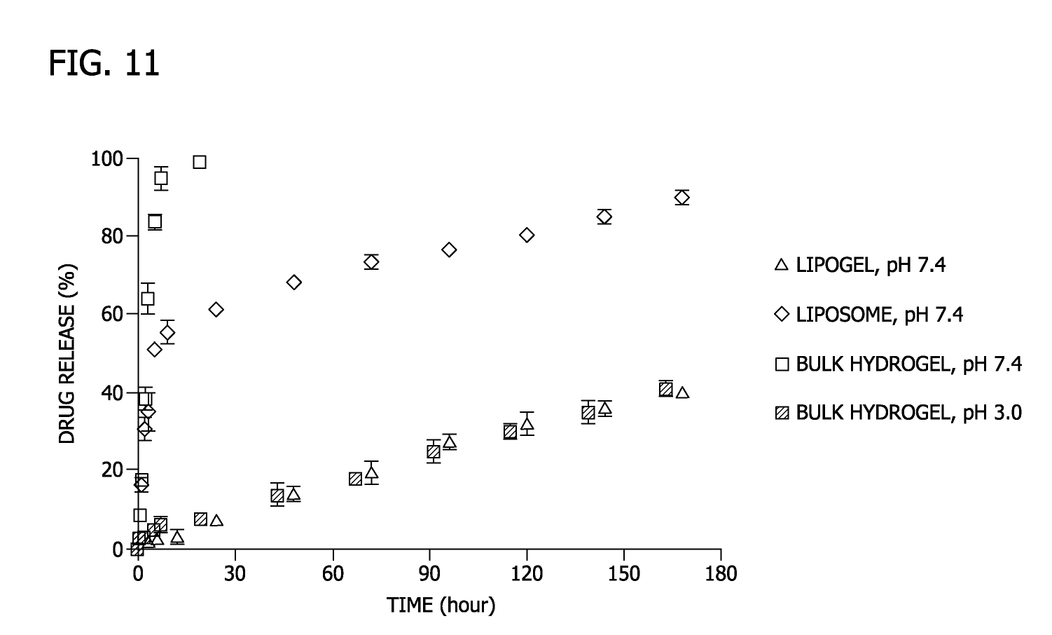

The release profiles of 17-DMAPG loaded lipogels, liposomes and bulk hydrogels are depicted in FIG. 11. At pH 7.4, both bulk hydrogel and liposomes exhibited burst release within the first 6 hours, by which time bulk hydrogel released about 90% loaded drug, while liposome released about 50%. In contrast, lipogels of the present disclosure displayed no burst effect and exhibited sustained release of 17-DMAPG over the 7 day drug release study. The accelerated release of 17-DMAPG from bulk hydrogel at pH 7.4 was probably due to the swelling of hydrogel matrix as a result of de-protonation on carboxylic acid groups at neutral pH. In order to further confirm this hypothesis, drug loaded bulk hydrogel was immersed in pH 3.0 acetate buffer, under which pH the PAA hydrogel matrix would not swell. Indeed, drug release from bulk hydrogel was drastically slowed down to almost the same level with lipogel drug release at pH 7.4. This experiment indicated that a low swelling ratio of the hydrogel played a role in retaining drug substance within the cross-linked polymer matrix, and the sustained release profile of lipogels may be due to the low swelling of hydrogel matrix covered and protected by the lipid-bilayer.

### Example 6

In this Example, the cytotoxicity of free 17-DMAPG and lipogel encapsulated 17-DMAPG was tested.

For cell culture, cells were maintained at 37° C., 5% CO₂ and 95% humidity in Dulbecco's modified Eagle's medium (DMEM) supplemented with 10% (v/v) heat inactivated fetal bovine serum (FBS), 2 mM L-glutamine and 100 unit/mL penicillin and 1000 μg/mL streptomycin. Cytotoxicity of 17-DMAPG loaded lipogels was assessed in human prostate PC-3 cancer cells and breast carcinoma MDA-MB-231 cells by resazurin-based metabolic assay. Briefly, cells were seeded in 96-well plates with 5,000 cells per well and allowed to adhere for 24 hours prior to the assay. Cells were exposed to various doses (0-35 μM on 17-DMAPG basis) of free 17-DMAPG, lipogel loaded 17-DMAPG and lipogel alone for 24 hours at 37° C., followed by washing with PBS, and maintaining in DMEM medium with 10% FBS for additional 24 hours. 10 μl of resazurin PBS solution (440 μM) was added into each well and incubated for a further 2 hours. The fluorescence was measured with a microplate reader (SpectraMax Gemini XS, Molecular Devices) with excitation at 560 nm and emission at 590 nm using wells without cells as blanks. The net fluorescent emission was taken as index of cell viability. The reading taken from the wells with cells cultured with control medium was used as 100% viability. The cell viability was calculated as Eₛₐₘₚₗₑ/E_{control}×100%. Based on the results of the test, the IC₅₀ values were calculated on GraphPad Prism 5 Software (San Diego Calif., USA).

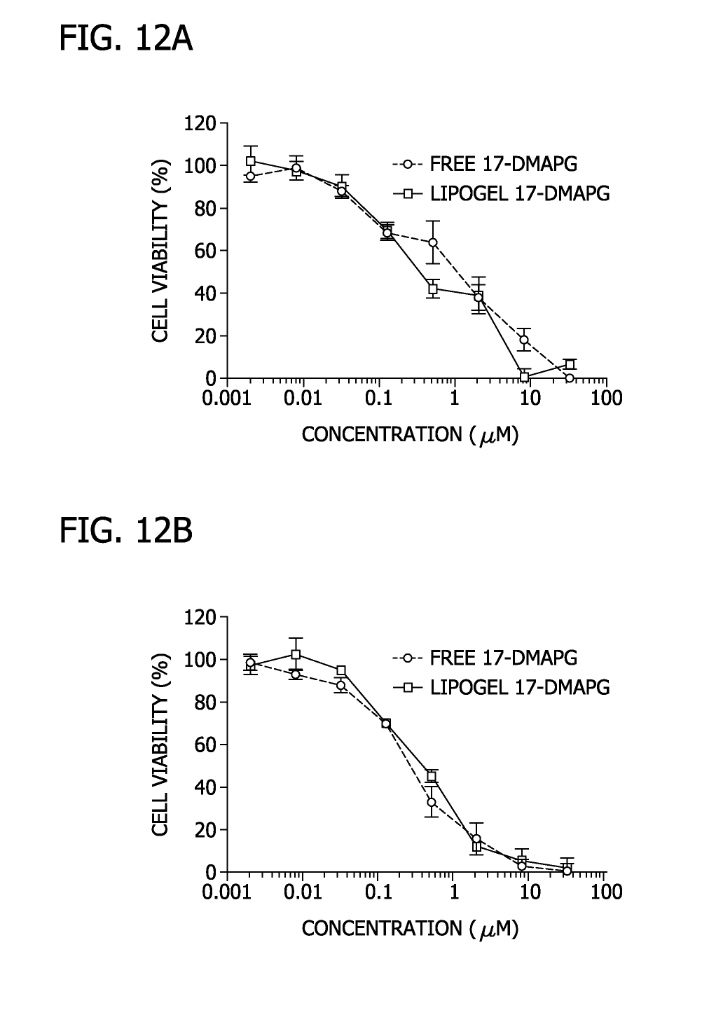

As depicted in FIGS. 12A and 12B, the lipogel formulation retained approximately the same level of cytotoxicity to both cell lines as compared to free drug with IC₅₀ at submicromolar level. IC₅₀ values are listed in table 1, below.

TABLE 1
In vitro cytotoxicity of lipogel 17-DMAPG

| | IC₅₀ (17-DMAPG equivalents in μM) | |
|---|---|---|
| Sample | PC-3 | MDA-MB-231 |
| Free 17-DMAPG | 0.807 | 0.152 |
| Lipogel 17-DMAPG | 0.466 | 0.330 |

As depicted in FIGS. 12A and 12B, the lipogel formulation retained approximately the same level of cytotoxicity to both cell lines as compared to free drug with IC₅₀ at submicromolar level. IC₅₀ values are listed in table 1, below.

What is claimed is:

1. A drug delivery system comprising a lipogel and at least one active agent.

2. The drug delivery system of claim 1, wherein the drug delivery system is a sustained release drug delivery system.

3. The drug delivery system of claim 1, wherein the lipogel comprises liposomes selected from the group consisting of dipalmitoylphosphatidylcholine, egg yolk L-α-phosphatidylcholine, 1,2-dimyristoyl-sn-glycero-3-phosphatidylcholine, 1,2-dipalmitoyl-sn-glycero-3-phosphatidylcholine, 1,2-dioleoyl-sn-glycero-3-phosphatidylcholine, 1,2-distearoyl-sn-glycero-3-phosphatidylcholine, 1,2-dilauroyl-sn-glycero-3-phosphatidylcholine, 1,2-dioleoyl-sn-glycero-3-phosphaethanolamine, 1-palmitoyl-2-oleoyl-sn-glycero-3-phosphaethanolaine, 1,2-dimyristoyl-sn-glycero-3-phosphaethanollamine, 1,2-dipalmitoyl-sn-glycero-3-phosphaethanolamine, 1,2-distearoyl-sn-glycerol-3-phosphaethanolamine, and combinations thereof.

4. The drug delivery system of claim 1, wherein the lipogel comprises a water soluble hydrogel.

5. The drug delivery system of claim 4, wherein the hydrogel is selected from the group consisting of acrylic acid, vinylsulfonic acid, 2-suloethylmethacrylate, 2-sulfoethyl acrylate, 2-(dimethylamino)ethyl methacrylate, 2-(dimethylamino)ethyl acrylate, 2-(diethylamino)ethyl acrylate, and combinations thereof

6. The drug delivery system of claim 4, wherein the hydrogel further comprises a cross-linker.

7. The drug delivery system of claim 6, wherein the cross-linker is selected from the group consisting of N,N′-methylenebis(acrylamide), poly(ethylene glycol)divinyl ether, divinyl benzene, N,N′-(1,2-dihydroxyethylene)bisacrylamide, ethylene glycol diacrylate, di(ethylene glycol)diacrylate, tri(ethylene glycol)diacrylate, tetra(ethylene glycol)diacrylate, ethylene glycol dimethacrylate, di(ethylene glycol)dimethacrylate, tri(ethylene glycol)dimethacrylate, tetra(ethylene glycol)dimethacrylate, pentaerythritol triacrylate, and combinations thereof.

8. The drug delivery system of claim 4, wherein the hydrogel further comprises an initiator.

9. The drug delivery system of claim 8, wherein the initiator is selected from the group consisting of 2,2-diethoxyacetophenone, 2,2-dimethoxy-2-phenylacetophenone, di-t(tertiary)-butylperoxide, azobisisobutyronitrile, 1-[4-(2-hydroxyethoxy)phenyl]-2-hydroxy-2-methyl-1-propan-1-one, 2-hydroxy-2-methylpropiophenone, and 2-hydroxy-4′-(2-hydroxyethoxy)-2-methylpropiophenone, ammonium persulfate, potassium persulfate, 1,1′-azobis(cyclohexanecarbonitrile), 2,2′-azobis 2-methyl-N-(2-hydroxyethyl)propionamide, 2,2′-azobis(2-amidinopropane)dihydrochloride, 4,4′-azobis(4-cyanovaleric acid), and combinations thereof.

10. A method for preparing a drug delivery system, the method comprising:
mixing a hydrogel precursor with a liposome to form a hydrogel precursor-liposome suspension;
extruding the hydrogel precursor-liposome suspension;
adding a free radical scavenger to the hydrogel precursor-liposome suspension;
treating the hydrogel precursor-liposome suspension to polymerize the hydrogel precursor encapsulated by the liposome to form a lipogel; and
incubating the lipogel with at least one active agent.

11. The method of claim 10, wherein the free radical scavenger is selected from the group consisting of ascorbic acid, uric acid, glutathione, melatonin, vitamin E and combinations thereof.

12. The method of claim 10, wherein the liposome is selected from the group consisting of dipalmitoylphosphatidylcholine, egg yolk L-α-phosphatidylcholine, 1,2-dimyristoyl-sn-glycero-3-phosphatidylcholine, 1,2-dipalmitoyl-sn-glycero-3-phosphatidylcholine, 1,2-dioleoyl-sn-glycero-3-phosphatidylcholine, 1,2-distearoyl-sn-glycero-3-phosphatidylcholine, 1,2-dilauroyl-sn-glycero-3-phosphatidylcholine, 1,2-dioleoyl-sn-glycero-3-phosphaethanolamine, 1-palmitoyl-2-oleoyl-sn-glycero-3-phosphaethanolaine, 1,2-dimyristoyl-sn-glycero-3-phosphaethanollamine, 1,2-dipalmitoyl-sn-glycero-3-phosphaethanolamine, 1,2-distearoyl-sn-glycerol-3-phosphaethanolamine, and combinations thereof.

13. The method of claim 10, wherein the liposome is in the form of a liposome film.

14. The method of claim 10, wherein the method further comprises mixing a cholesterol with the liposome prior to mixing the liposome with the hydrogel precursor.

15. The method of claim 10, wherein the hydrogel precursor comprises a water soluble monomer selected from the group consisting of acrylic acid, vinylsulfonic acid, 2-suloethylmethacrylate, 2-sulfoethyl acrylate, 2-(dimethylamino)ethyl methacrylate, 2-(dimethylamino)ethyl acrylate, 2-(diethylamino)ethyl acrylate, and combinations thereof.

16. The method of claim 15, wherein the method further comprises mixing a cross-linker with the water soluble monomer.

17. The method of claim 15, wherein the method further comprises mixing an initiator with the water soluble monomer.

18. The method of claim 17, wherein the initiator is selected from the group consisting of 2,2-diethoxyacetophenone, 2,2-dimethoxy-2-phenylacetophenone, di-t(tertiary)-butylperoxide, azobisisobutyronitrile, 1-[4-(2-hydroxyethoxy)phenyl]-2-hydroxy-2-methyl-1-propan-1-one, 2-hydroxy-2-methylpropiophenone, and 2-hydroxy-4′-(2-hydroxyethoxy)-2-methylpropiophenone, ammonium persulfate, potassium persulfate, 1,1′-azobis(cyclohexanecarbonitrile), 2,2′-azobis 2-methyl-N-(2-hydroxyethyl)propionamide, 2,2′-azobis(2-amidinopropane)dihydrochloride, 4,4′-azobis(4-cyanovaleric acid), and combinations thereof.

19. The method of claim 10, wherein the at least one active agent is selected from the group consisting of a drug, a nutraceutical agent, and a pharmaceutical agent.

20. A method for preparing a drug delivery system, the method comprising:
mixing a hydrogel precursor with a liposome to prepare a hydrogel precursor-liposome suspension;
extruding the hydrogel precursor-liposome suspension;
removing un-encapsulated hydrogel precursor from the hydrogel precursor-liposome suspension;
treating the hydrogel precursor-liposome suspension to polymerize encapsulated hydrogel precursor to form a lipogel; and
incubating the lipogel with at least one active agent.
